(19) United States
(12) Patent Application Publication
(10) Pub. No.: US 2021/0085234 A1
(43) Pub. Date: Mar. 25, 2021

(54) BRAINWAVE HEADBAND STRUCTURE

(71) Applicant: Sabrina Fashion Industrial Corp., New Taipei City (TW)

(72) Inventor: Yu-Ming Hsu, New Taipei City (TW)

(73) Assignee: Sabrina Fashion Industrial Corp., New Taipei City (TW)

(21) Appl. No.: 16/579,865

(22) Filed: Sep. 24, 2019

(51) Int. Cl. A61B 5/00

(52) U.S. Cl. CPC A61B 5/4064; A61B 5/6803

(57) ABSTRACT

The disclosure provides a brainwave headband structure, which includes a body and a brainwave detection module, wherein the body is provided with an inner surface and an outer surface. The brainwave detection module is arranged on the outer surface of the body and is electrically connected with a first electrode, a second electrode and a third electrode. The first electrode is arranged on the inner surface of the body, and the second electrode and the third electrode are oppositely arranged at two sides of the body in a manner of simultaneously covering a part of the inner surface and a part of the outer surface. When a user wears the body on a head, the first electrode is in contact with a forehead of the user, and the second electrode and the third electrode are in contact with ears of the user or peripheral regions near the ears.

### BACKGROUND

### Technical Field

The disclosure relates to a brainwave headband structure, in particular, to a brainwave headband structure capable of stably detecting brainwaves and maintaining wearing comfort.

### Description of Related Art

In recent years, the research results on the brain have become more and more mature. In the field of cognitive neuroscience, a brainwave detection device helps to know the brain activity state of human beings and monitor mental operation state changes of the brain, such as concentration levels and relaxation levels.

The existing commercially available products can be divided into hard brainwave detection devices and soft brainwave headbands. The hard brainwave detection device causes discomfort in wearing. Particularly, during sleep, the head leans on a pillow, and the hard brainwave detection device often causes discomfort of a wearer or even influences the sleep. On the other hand, a third electrode of the existing commercially available soft brainwave headband is generally designed as an ear clamp, and the ear clamp may easily fall off due to pull by a foreign object, further causing inconvenience in use or even influencing the detection results.

Based on the above, developing a brainwave detection device capable of stably detecting brainwaves and maintaining wearing comfort is an important subject to be researched at present.

### SUMMARY

The disclosure provides a brainwave headband structure, which allows a user to freely select two wearing modes, is capable of stably detecting brainwaves and maintaining wearing comfort, and solves the issues of poor electric contact and inconvenience in use.

The brainwave headband structure provided by the disclosure includes a body and a brainwave detection module. The body is provided with an inner surface and an outer surface. The brainwave detection module is arranged on the outer surface of the body, and is electrically connected with a first electrode, a second electrode and a third electrode. The first electrode is arranged on the inner surface of the body; the second electrode and the third electrode are oppositely arranged at two sides of the body in a manner of simultaneously covering a part of the inner surface and a part of the outer surface. When a user wears the body on a head, the first electrode is in contact with a forehead of the user, and the second electrode and the third electrode are in contact with ears of the user or peripheral regions near the ears.

In one embodiment of the disclosure, the second electrode and the third electrode are in contact with auricle regions or upper regions of the ears of the user.

In one embodiment of the disclosure, materials of the first electrode, the second electrode and the third electrode include conductive cloth or conductive ink.

In one embodiment of the disclosure, a sponge layer is arranged between the body and each of the first electrode, the second electrode and the third electrode.

In one embodiment of the disclosure, the brainwave headband structure further includes an adjusting element connected with the body.

In one embodiment of the disclosure, a distance from a center of the first electrode to a center of the second electrode is smaller than a distance from the center of the first electrode and a center of the third electrode.

In one embodiment of the disclosure, a ratio of the distance from the center of the first electrode to the center of the second electrode to the distance from the center of the first electrode and the center of the third electrode is 1:2 to 2:3.

In one embodiment of the disclosure, a length of the body is 40 cm to 65 cm, and a width of the body is 2 cm to 6 cm.

In one embodiment of the disclosure, a length of the first electrode is 1 cm to 10 cm, and a width of the first electrode is 1 cm to 4 cm.

In one embodiment of the disclosure, lengths of the second electrode and the third electrode are 1 cm to 20 cm, and widths of the second electrode and the third electrode are 1 cm to 8 cm.

Based on the above, in the brainwave headband structure provided by the disclosure, the second electrode and the third electrode simultaneously cover a part of the inner surface and a part of the outer surface of the body. Therefore, when the user wears the body on the head, it is possible to select between the wearing mode of contacting the second electrode and the third electrode with the ears of the user and the wearing mode of contacting them with the peripheral regions near the ears. In both modes, it is possible to stably detect signals and meanwhile maintain wearing comfort to prevent issues of poor electric contact and inconvenience in use.

In order to make the aforementioned and other objectives and advantages of the disclosure more comprehensible, embodiments accompanied with figures are described in detail below.

### BRIEF DESCRIPTION OF THE DRAWINGS

FIG. 1 is a schematic perspective view of a brainwave headband structure according to a first embodiment of the disclosure.

FIG. 2 is a schematic expanded view of the interior of the brainwave headband structure according to the first embodiment of the disclosure.

FIG. 3 is a schematic expanded view of the brainwave headband structure according to the first embodiment of the disclosure.

FIG. 4 and FIG. 5 are schematic wearing views of the brainwave headband structure according to the first embodiment of the disclosure.

FIG. 6 and FIG. 7 are schematic perspective views of a brainwave headband structure according to a second embodiment of the disclosure.

### DESCRIPTION OF THE EMBODIMENTS

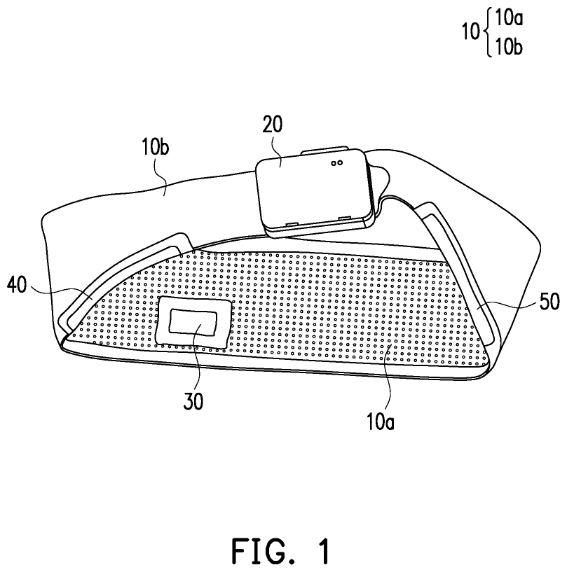

Referring to FIG. 1, the brainwave headband structure provided by the disclosure includes a body 10 and a brainwave detection module 20. The body 10 is provided with an inner surface 10a and an outer surface 10b. The brainwave detection module 20 is arranged on the outer surface 10b of the body 10, and is electrically connected with a first electrode 30, a second electrode 40 and a third electrode 50. The brainwave detection module 20, for example, is bonded on the body 10 through hot pressing adhesives. The first electrode 30 is arranged on the inner surface 10a of the body 10, and the second electrode 40 and the third electrode 50 are oppositely arranged at the two sides of the body 10 in a manner of simultaneously covering a part of the inner surface 10a and a part of the outer surface 10b.

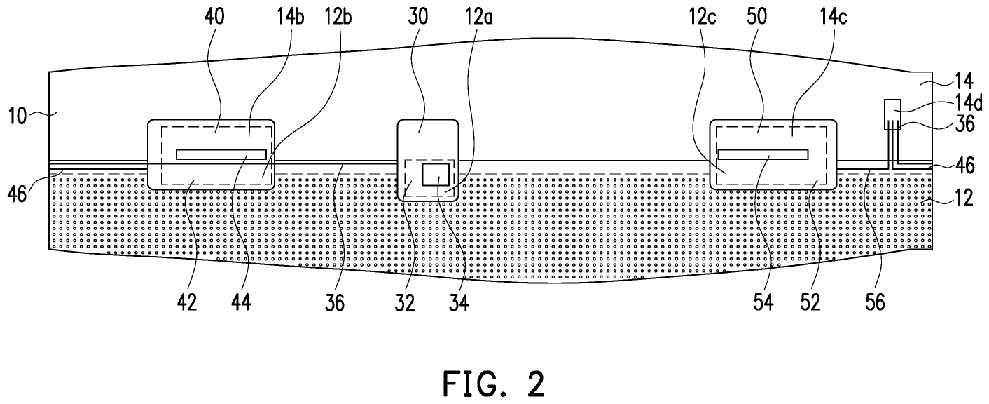

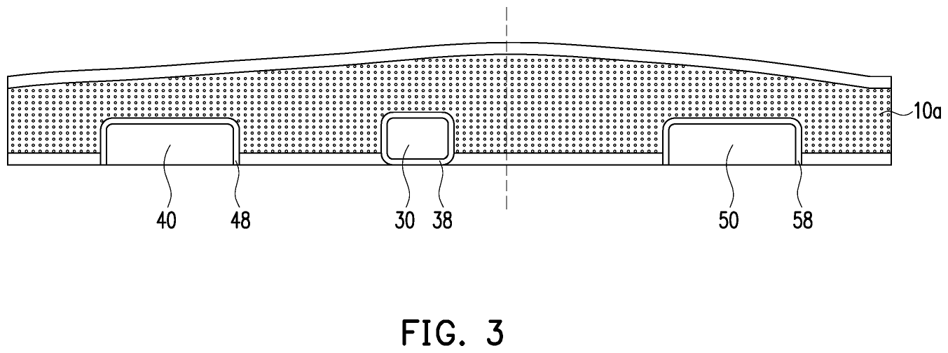

Referring to FIG. 2, the body 10 may be formed of an inner cloth 12 and an outer cloth 14. The inner cloth 12 and the outer cloth 14, for example, are bonded through hot pressing adhesives, wherein the inner cloth 12 is correspondingly provided with open holes 12a, 12b and 12c in positions corresponding to the first electrode 30, the second electrode 40 and the third electrode 50, the outer cloth 14 is correspondingly provided with open holes 14b and 14c in positions corresponding to the second electrode 40 and the third electrode 50, and the open holes 12b and 12c communicate with the open holes 14b and 14c. When the inner cloth 12 and the outer cloth 14 are folded along a dotted line in FIG. 2 to be assembled, the brainwave headband structure shown in FIG. 1 and FIG. 3 is formed, wherein the inner cloth 12 forms the inner surface 10a of the body 10, and the outer cloth 14 forms the outer surface 10b of the body 10.

Referring to FIG. 1, FIG. 2 and FIG. 3 together, materials of the first electrode 30, the second electrode 40 and the third electrode 50 include conductive cloth or conductive ink. As shown in FIG. 2, the first electrode 30, the second electrode 40 and the third electrode 50 are respectively formed by conductive clothes 32, 42 and 52, but the disclosure is not limited thereto, and the first electrode 30, the second electrode 40 and the third electrode 50 can also be prepared from conductive ink according to practical requirements. In addition, as shown in FIG. 2, sponge layers 34, 44 and 54 are respectively arranged between the body 10 and each of the first electrode 30, the second electrode 40 and the third electrode 50 for improving the comfort level and fitness level of the user at the time of wearing the body on the head. As shown in FIG. 3, decorative adhesives 38, 48 and 58 are respectively arranged on the outer edges of the first electrode 30, the second electrode 40 and the third electrode 50. In more details, the length of the body 10, for example, is 40 to 65 cm, and the width, for example, is 2 to 6 cm. The length of the first electrode 30, for example, is 1 to 10 cm, and the width, for example, is 1 to 4 cm. The lengths of the second electrode 40 and the third electrode 50, for example, are 1 to 20 cm, and the widths, for example, are 1 to 8 cm.

Referring to FIG. 2, the open holes 12a, 12b, 12c, 14b and 14c are formed in the outer cloth 14 and the inner cloth 12 through laser cutting, the conductive clothes 32, 42 and 52 are placed, and the sponge layers 34, 44 and 54 are wrapped in the conductive clothes 32, 42 and 52, which are then bonded onto the outer cloth 14 and the inner cloth 12 through hot pressing adhesives. The first electrode 30, the second electrode 40 and the third electrode 50 may be respectively bonded with conducting wires 36, 46 and 56 through UV adhesives, or the conducting wires 36, 46 and 56 may be respectively connected with the conductive clothes 32, 42 and 52 through metal hooks. The conducting wires 36, 46 and 56 are gathered to another open hole 14d to be electrically connected with the brainwave detection module 20. The conducting wires 36, 46 and 56, for example, are shielding wires, which need to be provided with insulation surfaces, and for example, are electric wires, enameled wires or conductive elastic yarns.

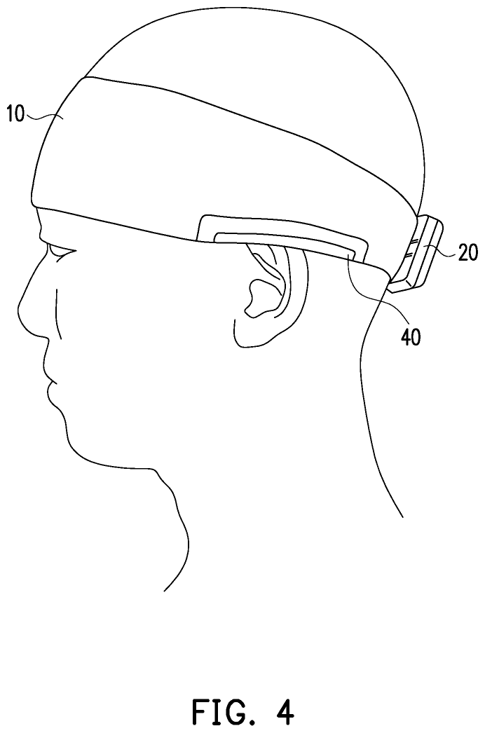

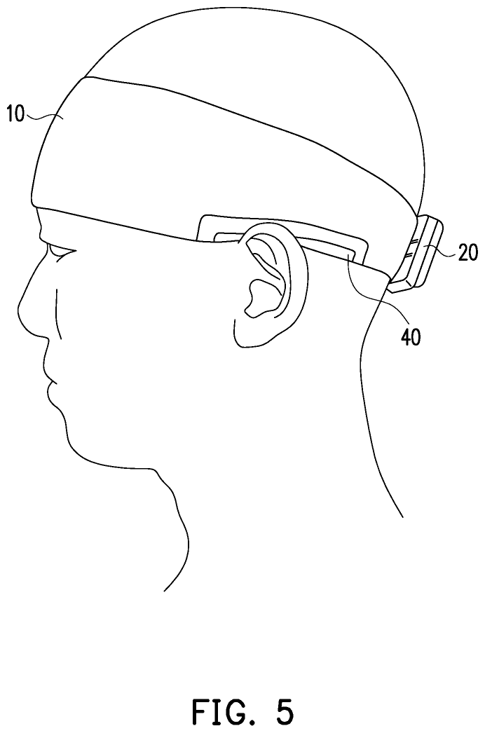

Referring to FIG. 1, FIG. 3, FIG. 4 and FIG. 5, when the user wears the body 10 on the head, the first electrode 30 is in contact with the forehead of the user, and the second electrode 40 and the third electrode 50 are in contact with the ears of the user or the peripheral regions near the ears. In more details, as shown in FIG. 4, the second electrode 40 and the third electrode 50 are in contact with the auricle regions of the ears of the user; and as shown in FIG. 5, the second electrode 40 and the third electrode 50 are in contact with the regions above the ears of the user. The second electrode 40 and the third electrode 50 of the disclosure are oppositely arranged at the two sides of the body 10 in a manner of simultaneously covering a part of the inner surface 10a and a part of the outer surface 10b, so that when the user wears the body 10 on the head, the second electrode 40 and the third electrode 50 are in contact with the auricle regions of the ears of the user or the upper regions to stably detect signals and meanwhile maintain wearing comfort. As shown in FIG. 4 and FIG. 5, the brainwave detection module 20 may be arranged, for example, at the back side of the head of the user near the neck, but the disclosure is not limited thereto, and the brainwave detection module 20 can also be arranged in the forehead position of the user.

Referring to FIG. 1, FIG. 3, FIG. 4 and FIG. 5, when the user wears the body 10 on the head, the second electrode 40 and the third electrode 50 are respectively in contact with the left ear and the right ear of the user. At this time, the distance from the center of the first electrode 30 to the center of the second electrode 40, for example, is smaller than the distance from the center of the first electrode 30 to the center of the third electrode 50. In other words, the second electrode 40 in contact with the left ear of the user is closer to the first electrode 30 than the third electrode 50. In more details, the ratio of the distance from the center of the first electrode 30 to the center of the second electrode 40 to the distance from the center of the first electrode 30 to the center of the third electrode 50 is 1:2 to 2:3.

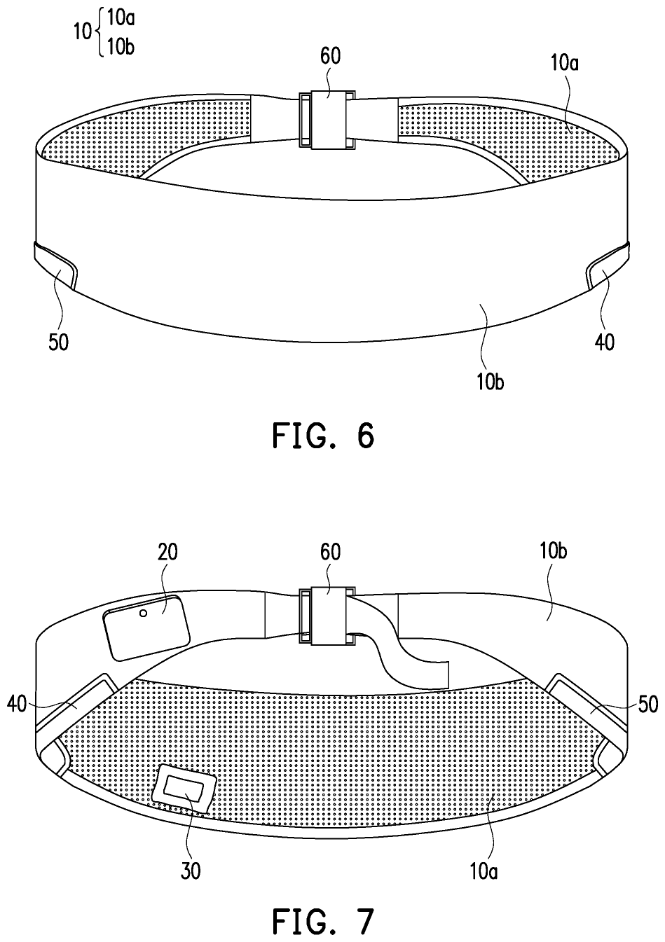

Referring to FIG. 6 and FIG. 7, the second embodiment shown in FIG. 6 and FIG. 7 is similar to the first embodiment shown in FIG. 1 to FIG. 5, so identical components are expressed by identical numbers, and the descriptions thereof are omitted herein. The difference between the first embodiment and the present embodiment lies in that the brainwave headband structure in the present embodiment further includes an adjusting element 60 connected to the body 10. The adjusting element 60, for example, includes a male clamp buckle and a female clamp buckle, which are mutually matched. The circumference of the body 10 can be adjusted after the body 10 is arranged to penetrate through either the male clamp buckle or the female clamp buckle.

Based on the above, in the brainwave headband structure provided by the disclosure, the second electrode and the third electrode simultaneously covers a part of the inner surface and a part of the outer surface of the body. Therefore, when the user wears the body on the head, it is possible to select between the wearing mode of contacting the second electrode and the third electrode with the ears of the user and the wearing mode of contacting them with the peripheral regions near the ears. In both modes, it is possible to stably detect signals and meanwhile maintain wearing comfort. In addition, the sponge layers are arranged between the body and each of the first electrode, the second electrode and the third electrode for improving the comfort level and fitness level of the user at the time of wearing the body on the head.

It should be finally noted that the above embodiments are merely intended for describing the technical solutions of the disclosure rather than limiting the disclosure. Although the disclosure is described in detail with reference to the foregoing embodiments, persons of ordinary skill in the art should understand that they can still make modifications to the technical solutions described in the foregoing embodiments or make equivalent substitutions to some technical features thereof, without departing from scope of the technical solutions of the embodiments of the disclosure.

What is claimed is:

1. A brainwave headband structure, comprising:
a body, comprising an inner surface and an outer surface; and
a brainwave detection module, arranged on the outer surface of the body, and electrically connected with a first electrode, a second electrode and a third electrode,
wherein the first electrode is arranged on the inner surface of the body, the second electrode and the third electrode are oppositely arranged at two sides of the body in a manner of simultaneously covering a part of the inner surface and a part of the outer surface, and
when a user wears the body on a head, the first electrode is in contact with a forehead of the user, and the second electrode and the third electrode are in contact with ears of the user or peripheral regions near the ears.

2. The brainwave headband structure according to claim 1, wherein the second electrode and the third electrode are in contact with auricle regions or upper regions of the ears of the user.

3. The brainwave headband structure according to claim 1, wherein materials of the first electrode, the second electrode and the third electrode comprise conductive cloth or conductive ink.

4. The brainwave headband structure according to claim 1, wherein a sponge layer is arranged between the body and each of the first electrode, the second electrode and the third electrode.

5. The brainwave headband structure according to claim 1, further comprising an adjusting element connected to the body.

6. The brainwave headband structure according to claim 1, wherein a distance from a center of the first electrode to a center of the second electrode is smaller than a distance from the center of the first electrode to a center of the third electrode.

7. The brainwave headband structure according to claim 6, wherein a ratio of the distance from the center of the first electrode to the center of the second electrode to the distance from the center of the first electrode to the center of the third electrode is 1:2 to 2:3.

8. The brainwave headband structure according to claim 1, wherein a length of the body is 40 cm to 65 cm, and a width of the body is 2 cm to 6 cm.

9. The brainwave headband structure according to claim 1, wherein a length of the first electrode is 1 cm to 10 cm, and a width of the first electrode is 1 cm to 4 cm.

10. The brainwave headband structure according to claim 1, wherein lengths of the second electrode and the third electrode are 1 cm to 20 cm, and widths of the second electrode and the third electrode are 1 cm to 8 cm.
